(19) United States
(12) Patent Application Publication
(10) Pub. No.: US 2026/0158189 A1
(43) Pub. Date: Jun. 11, 2026

(54) EXTRACELLULAR VESICLES-MODIFIED METAL-IMPLANTS WITH IMMUNOREGULATORY AND OSTEOINDUCTIVE FUNCTIONS AND PREPARATION METHOD THEREOF

(71) Applicant: JIANGSU UNIVERSITY, Zhenjiang (CN)

(72) Inventors: Guoqing PAN, Zhenjiang (CN); Dechun GENG, Zhenjiang (CN); Gaoran GE, Zhenjiang (CN); Shun GUO, Zhenjiang (CN); Miao WANG, Zhenjiang (CN)

(73) Assignee: JIANGSU UNIVERSITY, Zhenjiang (CN)

(21) Appl. No.: 18/710,213

(22) PCT Filed: Oct. 16, 2023
(86) PCT No.: PCT/CN2023/124711

§ 371 Date: May 15, 2024

(30) Foreign Application Priority Data
Sep. 14, 2023 (CN) 202311186758.4

(51) Int. Cl. A61L 27/28; A61L 27/06

(52) U.S. Cl. CPC A61L 27/28; A61L 27/06; A61L 2400/18; A61L 2420/02

(57) ABSTRACT

Extracellular vesicles-modified metal-implants with immunoregulatory functions, and a preparation method and use thereof are provided. A mussel derived adhesion peptide containing catechol group was designed by combining metal-phenol coordination chemistry of mussel-mimetic adhesion molecules with bio-orthogonal click chemical modification strategy. The metal-implants modified by extracellular vesicles can be obtained. In the process of modification, the extracellular vesicles (such as exosomes) can be modified on the surface of medical metal implant materials through the strong catecol/titanium (Ti) coordination interaction to modify the metal Ti implant and connect it with azido-modified extracellular vesicles. Results show that the osseointegration efficiency of the implant material surface can be improved through its immunomodulatory function and osteoinductive activity.

### CROSS REFERENCE TO THE RELATED APPLICATIONS

This application is the national phase entry of International Application No. PCT/CN2023/124711, filed on Oct. 16, 2023, which is based upon and claims priority to Chinese Patent Application No. CN202311186758.4, filed on Sep. 14, 2023, the entire contents of which are incorporated herein by reference.

### TECHNICAL FIELD

The present belongs to the field of biomedical materials, and specifically relates to an extracellular vesicles-modified metal-implant with immunoregulatory and osteoinductive dual-functions, and a preparation method and a use thereof.

### BACKGROUND

Extracellular vesicles (such as exosomes) are vesicles containing a phospholipid bilayer structure that can be secreted by almost all types of cells. They play a pivotal role in cell therapy. They can participate in many pathophysiological processes such as inflammation, autoimmune response, endothelial dysfunction/injury, procoagulation, angiogenesis and intimal hyperplasia, osteogenic differentiation. Usually, extracellular vesicles carry various functional groups such as cytokines, growth factors, signaling lipids, DNA and regulatory miRNA. Among them, extracellular vesicles derived from mesenchymal stem cells (MSC-EVs) during early osteogenic differentiation have shown positive effects on anti-inflammation, cell adhesion and osteogenic differentiation, and are ideal regulatory substances for osseointegration. Compared with other stem cells, protein drugs and synthetic chemicals, the natural multi-components of MSC-EVs can induce complex and long-lasting cellular responses in a safe, low-cost and efficient manner, without the limitation of immune and biological toxicity, which has obvious advantages for improving the surface osseointegration of prostheses.

Prosthesis implantation is one of the most successful treatment strategies for bone and joint functional reconstruction. However, the failure rate of orthopedic implants is increased due to the immune response after prosthesis implantation. The immune response can hind osseointegration and then affect the effect of osseointegration. During prosthesis implantation, biological quality of bone is maintained by stone remodeling. And the bone metabolism and immuno-inflammatory response are the major factors in the process of stone remodeling. The main characters of bone metabolism are reduction of stone formation and the circulating levels of biochemical markers of bone formation. In addition to bone metabolism, the more important factor affecting osteogenesis is the inflammatory immune response. It is reported that inflammatory cytokines released by immune cells can be detected in patients with prosthesis implantation, in which macrophages play a key role. After prosthesis implantation, the local immune response will be aggravated by exogenous biomaterials, and macrophages will be recruited to the implant surface in several hours after surgery then trigger an early inflammatory cascade. Long-term inflammatory exposure can cause the formation of annulus fibrosum, which develops into a key factor affecting the survival rate of prosthesis and becomes a barrier to osseointegration. The prolonged inflammatory exposure is also harmful for the activity of MSC and the osteogenic differentiation ability. Until now, it is still the severe challenge for the integration and bone remodeling between prosthesis and bone.

Recent studies showed that the integration should be improved by promoting bone formation through immunomodulation. However, the flexible modification strategies based on cells, vesicles or macromolecular bioactive substances still need to be explored. Especially, the process of modifying medical implants with biomass to obtain high-performance prostheses is seriously insufficient. The novel and effective strategies for implant surface modification with MSC-EVs to improve osseointegration on the surface of prostheses is also urgent needed.

### SUMMARY

To solve the problem in the prior art, the present disclosure provides a preparation method and a use of extracellular vesicles-modified metal-implants with immunoregulatory and osteoinductive dual-functions. The mussel-derived peptide ((DOPA)ₓ-PEG₅,-DBCO) synthesized by biologically clickable groups (DBCO). The peptides adhere to the surface of Ti implant and can click with the azido group linked to the extracellular vesicles. Ti implant is modified by synthetic peptides and clickable high active phospholipid poly (ethylene glycol) derivative 1,2-distearyl-sn-glycerol-3-hydroethanolamine phosphate azide (DSPE-PEG₂ₖ-Azido) linked to pre-differentiated MSC-extracellular vesicles. The extracellular vesicles-modified metal-implants with immunoregulatory and osteoinductive functions could modulate the macrophage polarization in vitro and in vivo, then promote periprosthetic osseointegration.

To achieve the above objective, the following technical solutions are chosen:

The present disclosure provides an extracellular vesicles-modified metal-implants with dual-functions (immunoregulatory and osteoinductive functions). Mussel derived adhesion peptides and azido extracellular vesicles were chemically modified on the surface of Ti based materials to obtain extracellular vesicles-modified Ti implant with the dual-functions of immunoregulation and osteoinduction.

Preferably, the extracellular vesicles-modified medical implant is a Ti material modified with the clickable mussel-derived peptide (DOPA)x-PEGs- DBCO on its surface and it could react with the Azido groups linked to extracellular vesicles. The modification of extracellular vesicles could realize by cycloaddition bioorthogonal click chemistry between azido extracellular vesicles (DSPE-PEG₂ₖ-Azido) and (DOPA)ₓ-PEG₅-DBCO stable and nontoxic.

In a particular embodiment, the present disclosure provides a method for preparing an extracellular vesicles-modified metal-implants with dual functions of immunomodulation and osteoinductive, including the following steps:
(1) Designing and preparing mussel-derived adhesion peptides

Solid phase peptide synthesis is chosen to prepare mussel-derived peptide through mixing copper-free click chemical dibenzocyclooctyne (DBCO), acetone with Fmoc protected L-dopa amino acids (Fmoc-DOPA(acetonide)-OH), labels as (DOPA)ₓ-PEG₅-DBCO;
(2) Derived peptide-modification on the surface of medical implant

DOPA-implant is obtained immersing the medical implant into (DOPA)ₓ-PEG₅-DBCO solution obtained in step (1) by solution immersion method, and DOPA groups is adhered to the surface of implant through fully adhering.
(3) Azido modification on the surface of extracellular vesicles

Extracellular vesicles modified by azido is prepared by mixing extracellular vesicles (EV) with azide coupling reagent 1,2-distearacyl-SN-glycerol-3-hydroethanolamine phosphate (DSPE-PEG₂ₖ-Azido), and it is obtained by the combined of EV and DSPE, labels as EV-DSPE-PEG₂ₖ-Azido.
(4) Click chemical modification of extracellular vesicles on the surface of medical implant

The extracellular vesicles-modified metal-implants with immunoregulation and osteoinductive dual-functions is prepared by immersing the DOPA-implant in step (2) into the EV-DSPE-PEG₂ₖ-Azido solution. The specific loading of extracellular vesicles is realized by the Cycloaddition bioorthogonal click chemistry between azide groups and DBCO groups.

The brief structural formula of mussel-derived polypeptide sequences in step (1) is Ac-[(DOPA)-G]_{x/2}-K[(PEGs)-(Mpa)-(Mal-DBCO)]-[(DOPA)-G]_{x/2}, in which, x represents the repeat unit of DOPA amino acid, and it can be chosen from 2, 4, 6, and 8.

In detail, the mussel-derived peptides synthesized in step (1) contain glycine (G) or lysine (K) for interval, which spaces the L-dopa amino acids by a glycine or lysine (K). With the help of the side-chain amino group of lysine (K), the clickable diphenylcyclooctene group is linked by the bridging action of short-chain polyethylene glycol (PEG₅).

In the synthesis of the mussel-derived adhesion peptide described in step (1), the dopa groups in single peptide is spaced by a glycine. The overall chain length of mussel-derived peptides is 5, 9, 13 or 17 amino acid, and they are linked in linear.

The present invention uses acetone as a protective reagent to protect the catechol group in the L-DOPA amino acid (DOPA).

In the step (2), the medical implant is Ti alloy material, and is preferably Ti plate, Ti rod, or Ti nail. The concentration of (DOPA)x-PEG 5-DBCO solution is 0.005 mg/mL-1.000 mg/mL, and the time of full immersion is 12-36 h.

In the step (3), the quantity ratio of EV and DSPE-PEG2k-Azido is 1:100-1:10000.

In the step (4), the concentration of EV-DSPE-PEG2k-Azido solution is 0.01-1.0 mg/mL, and the time of immersion is 6-24 h.

Further, the present disclosure provides a use of an extracellular vesicles-modified medical implant with immunoregulation and osteoinductive dual-functions as described above in the field of preparing artificial prosthesis implant materials.

Preferably, the present disclosure provides a use of the extracellular vesicles-modified medical implant in the field of preparing artificial prosthesis implant materials for diabetic patients.

Compared with the prior art, the present disclosure has the following advantages.

Inspired by marine mussels, the present disclosure designs a mussel-derived adhesion peptide with catechol groups by combining metal-phenol coordination chemistry mimicking mussel adhesion molecules with a bioorthogonal click chemical modification strategy. The metal Ti implants were modified by catechol-titanium (Ti) coordination interaction, and then connected with the azido-modified extracellular vesicles to obtain the extracellular vesicles-modified medical implants with immunoregulation and osteoinduction dual-function. The extracellular vesicles-modified medical implants with the immunoregulation and osteoinduction dual-function could be modified on the surface of medical metal implants without any damage, and the implant materials could be endowed with immunomodulatory function and osteoinduction activity.

Mesenchymal stem cell-derived extracellular vesicles with multi-bioactive, early osteogenic differentiation are modified on the surface of bone implants by mimicry of mussel adhesion mechanisms and bioorthogonal click responses. It can regulate the polarization phenotype of macrophages and play an immune-osteogenic cascade to improve the performance of implants. It can reduce the inflammatory response in hyperglycemic microenvironment and reduce the expression of IL-1β, IL-6 and TNF-α related inflammatory factors. It also can promote the osteogenic differentiation ability of BMSC under high glucose microenvironment and promote periprosthetic osseointegration, then promote bone formation and increase bone mineral density in DM environment.

The obtained extracellular vesicles-modified medical implants have good biocompatibility, promote the polarization of macrophages to the anti-inflammatory M2 phenotype, and synergistically improve the induction of immune microenvironment at the bone-implant interface, the best osteogenic performance and osseointegration effect at the bone-implant interface. Comparing with the complexity and potential toxicity and damage of traditional chemical modifications, mussel-like adhesion and click modification strategies, preparation methods that are fast, mild and do not destroy the structural stability of extracellular vesicles, provides the new method for efficient modification of functional extracellular vesicles on the surface of medical metal implants. In particular, it has a profound impact on improving the survival rate of diabetic patients with prosthesis implantation, and has a broad prospect in the medical field.

### BRIEF DESCRIPTION OF THE DRAWINGS

FIG. 1 Identification of primary MSC markers by flow cytometry;

FIG. 2 Identification of extracellular vesicles: A images of NTA particle size analysis and transmission electron microscopy (TEM), and B identification of extracellular vesicles marker protein expression;

FIG. 3 Schematic illustration of molecular structure of mussel-derived peptide components: a: the representative mussel-derived peptides (DOPA)₆-PEG₅-DBCO, b: the molecular formula of DSPE-PEG₂ₖ-Azido, and c: molecular formula of cycloaddition reaction;

FIG. 4 HPLC and MS analysis of representative (DOPA)₆-PEG₅-DBCO;

FIG. 5 NMR analysis of DSPE-PEG₂ₖ-Azido;

FIG. 6 Images observed by fluorescence microscopy (FITC);

FIG. 7 AFM images of surface topography;

FIG. 8 XPS analysis of surface element composition;

FIG. 9 Water contact angle images (WCA);

FIG. 10 SEM of MSCs and RAW 264.7 cells;

FIG. 11 Plots of mean cell spread analysis and cell proliferation activity assay;

FIG. 12 Live/dead assay of MSCs and RAW 264.7 cells (**p<0.01);

FIG. 13 Fluorescence image of reactive oxygen species detection for osteogenesis of EV-DOPA;

FIG. 14 Quantitative flow cytometry analysis of reactive oxygen species for osteogenesis of EV-DOPA;

FIG. 15 Osteogenesis staining of EV-DOPA: (A) ALP staining and (B) ARS staining;

FIG. 16 ALP activity assay and ARS absorbance quantitative determination of osteogenesis of EV-DOPA;

FIG. 17 RT-qPCR results for osteogenesis-related gene expression of EV-DOPA;

FIG. 18 Immunofluorescence staining results of related proteins Runx2 and OCN expression;

FIG. 19 Schematic illustration of the experimental design by macrophage polarization;

FIG. 20 Co-immunofluorescence staining images of iNOS and F4/80 in macrophages;

FIG. 21 Co-immunofluorescence staining images of Arg-1 and F4/80 in macrophages;

FIG. 22 Mean fluorescence intensity analysis images of immunofluorescent staining;

FIG. 23 Flow quantitative analysis of M1-polarized phenotype CD86 in macrophages;

FIG. 24 Flow quantitative analysis of M2-polarized phenotype CD206 in macrophages;

FIG. 25 RT-qPCR of inflammatory cytokines;

FIG. 26 Expression analysis of inflammatory factors by high-throughput sequencing;

FIG. 27 Staining under EV-DOPA-regulated immune conditions: (A) ALP staining and (B) ABS staining;

FIG. 28 ALP activity assay and ARS absorbance quantitative determination of EV-DOPA-regulated immune conditions;

FIG. 29 RT-qPCR results for EV-DOPA-regulated immune conditions;

FIG. 30 Immunofluorescence staining results of related proteins Runx2 and OCN expression under EV-DOPA-regulated immune conditions;

FIG. 31 Blood glucose monitoring chart of DM rat model: A continuous blood glucose monitoring of various week-aging rats and B glucose tolerance test;

FIG. 32 Co-immunofluorescence staining and mean fluorescence intensity analysis images of CD86;

FIG. 33 Co-immunofluorescence staining and mean fluorescence intensity analysis images of CD206;

FIG. 34 Three-dimensional reconstruction images of bone mass and bone volume fraction analysis of normal rats and DM rats before and after titanium rod implantation: A before titanium rod implantation and B after titanium rod implantation;

FIG. 35 Micro-CT bone three-dimensional reconstruction image of EV-DOPA on periprosthetic osseointegration in DM rat;

FIG. 36 Bone volume fraction analysis, bone mineral density analysis and trabecular thickness analysis of EV-DOPA on periprosthetic osseointegration in DM rat;

FIG. 37 H&E staining of EV-DOPA on periprosthetic osseointegration in DM rat;

FIG. 38 Toluidine blue staining images;

FIG. 39 Analysis of fibrous layer thickness and bone-prosthesis contact ratio;

FIG. 40 Calcein fluorescence images of EV-DOPA on osteogenic markers of the prosthesis periphery in DM rats;

FIG. 41 Protein immunofluorescence staining images of OCN and COLIA1.

### DETAILED DESCRIPTION OF THE EMBODIMENTS

In order to enable those skilled in the art to understand the present disclosure comprehensively, the present disclosure will be further described below in conjunction with the accompanying drawings and specific examples, but the protection scope of the present disclosure is not limited thereto. The present invention describes generally and/or specifically the materials and test methods used in the test. Experimental methods without specific conditions specified in the following examples are usually tested under conventional conditions or under conditions suggested by the instruction. Reagents, biological materials, etc., used in the following embodiments are available from commercial sources unless otherwise specified.

Osteogenic induction medium was purchased from Shanghai Xiaopeng Biological Technology Co., LTD. Fmoc-DOPA(acetonide)-OH was purchased from Qianyao Biological Technology Co., LTD. DSPE-PEG2k-Azido was purchased from Xi'an Rayxi Biological Technology Co., LTD. ALP alkaline phosphatase staining kit was purchased from Biyuntian Biological Technology Co., LTD. NanoDrop-2000 was purchased from Thermo Fisher. PrimeScript RT Master Mix and Ex TaqTM were purchased from Takara. Primary antibody working solution, secondary antibody working solution, goat serum blocking solution, antibody I and antibody II were purchased from Abcam. SD rats were obtained from the Animal Laboratory Center of Soochow University.

The present disclosure provides a method for preparing an extracellular vesicles-modified metal-implants with dual functions of immunomodulation and osteoinductive, including the following steps:
(1) Designing and prepararing mussel-derived adhesion peptides

Solid phase peptide synthesis is chosen to prepare mussel-derived peptide through mixing copper-free click chemical dibenzocyclooctyne (DBCO), acetone with Fmoc protected L-dopa amino acids (Fmoc-DOPA(acetonide)-OH), labels as (DOPA)ₓ-PEG₅-DBCO. The brief structural formula of L-dopa amino acids sequences in step (1) is Ac-[(DOPA)-G]ₓ₂-K[(PEG₅)-(Mpa)-(Mal-DBCO)]-[(DOPA) -G]_{x/2}, in which, x represents the repeat unit of DOPA amino acid, and it can be chosen from 2, 4, 6, and 8. The overall chain length of mussel-derived peptides is 5, 9, 13 or 17 amino acid, and they are linked in linear.
(2) Peptide-modification on the surface of medical implant

DOPA-implant is obtained immersing the medical implant into (DOPA)-PEGs-DBCO solution with the concentration of 0.005 mg/mL-1.000 mg/mL obtained in step (1) by solution immersion method for 12 h-36 h, and DOPA groups is adhered to the surface of implant through fully adhering. The medical implant is Ti plate, Ti rod, Ti nail or other conventional Ti alloy prosthesis materials that can be used for implantation.
(3) Azido modification on the surface of extracellular vesicles

Extracellular vesicles modified by azido is prepared by mixing extracellular vesicles (EV) extracted from MSC with azide coupling reagent 1,2-distearacyl-SN-glycerol-3-hydroethanolamine phosphate (DSPE-PEG₂ₖ-Azido) in 1:100-1:10000, and it is obtained by the combined of EV and DSPE, labels as EV-DSPE-PEG₂ₖ-Azido.
(4) Click chemical modification of extracellular vesicles on the surface of medical implant

The extracellular vesicles-modified metal-implants with immunoregulation and osteoinductive dual-functions is prepared by immersing the DOPA-implant in step (2) into the EV-DSPE-PEG₂ₖ-Azido solution with the concentration of 0.01-1.0 mg/mL for 6 h-24 h. The specific loading of extracellular vesicles is realized by the Cycloaddition bioorthogonal click chemistry between azide groups and DBCO groups.

### Example 1: Extraction and Identification of Extracellular Vesicles Derived From Predifferentiated MSCs

6-week-old rats (200 g, SPF, Animal Laboratory Center of Soochow University) were sacrificed under anesthesia and primary bone marrow mesenchymal stem cells (BMSCs) extracted from the long bones of both hind limbs. After 3 days of osteogenic induction medium containing extracellular vesicles-free serum, the supernatant was collected and centrifuged to remove cell debris. Subsequently, extracellular vesicles were extracted by ultrafiltration method. Extracellular vesicles were tested by NTA particle size detection and expression of protein markers.

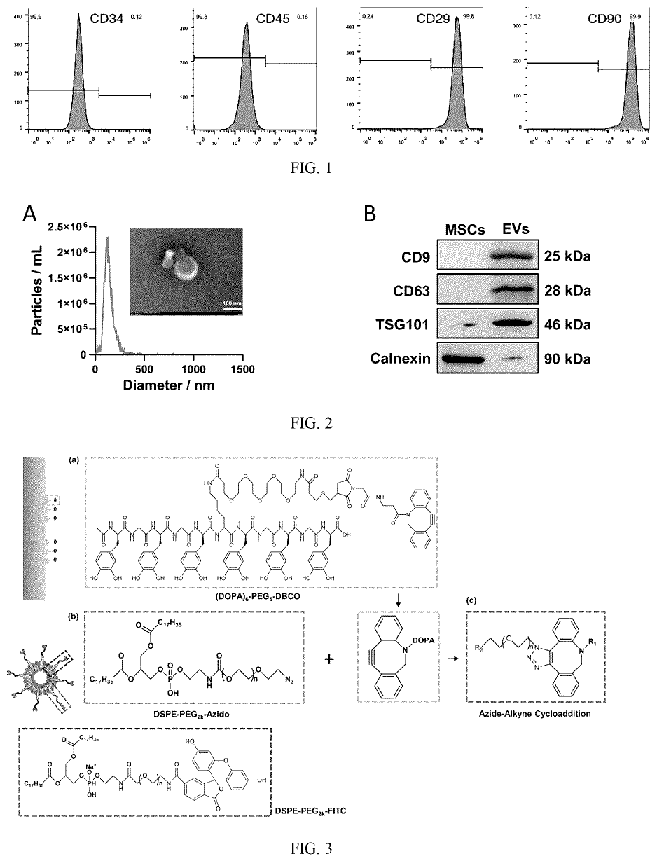

FIG. 1 is the identification of primary MSC markers by flow cytometry. FIG. 2 is the identification of extracellular vesicles. In the figure, A is the images of NTA particle size analysis and transmission electron microscopy (TEM), and B is the identification of extracellular vesicles marker protein expression. The results of flow cytometry showed that the negative of CD34, CD45 and the positive of CD9, CD63. The results of NTA particle size detection show that the diameter distribution of the extracted microvesicles is concentrated in 100-200 nm. The positive of extracellular vesicles markers CD9, CD63, TSG101 indicates that extracellular vesicles from primary BMSCs had been successfully obtained.

### Example 2: Preparation of EV-DOPA-Coated Modified Implants

### (1) Synthesis of Mussel-Derived Peptides

Solid phase peptide synthesis is chosen to prepare mussel-derived peptide through mixing copper-free click chemical dibenzocyclooctyne (DBCO), acetone with Fmoc protected L-dopa amino acids (Fmoc-DOPA(acetonide)-OH), labels as (DOPA)x-PEG5-DBCO, in which, x represents the repeat unit of DOPA amino acid. In detail, the mussel-derived peptides synthesized in step (1) contain glycine (G) or lysine (K) for interval, which spaces the L-dopa amino acids by a glycine or lysine (K). With the help of the side-chain amino group of lysine (K), the clickable diphenylcyclooctene group is linked by the bridging action of short-chain polyethylene glycol (PEG5). FIG. 3 shows the schematic illustration of molecular structure of mussel-derived peptide components, in which, a is the representative mussel-derived peptides (DOPA)₆-PEG₅-DBCO, b is the molecular formula of DSPE-PEG_{2K}-Azido, and c is the molecular formula of cycloaddition reaction. FIG. 4 is the HPLC and MS analysis of representative (DOPA)₆-PEG₅-DBCO. The results indicate that the purity of synthetic peptides is 98.35% which is consistent with the designed molecular weight.

### (2) Polypeptideization of Ti Alloy Implants

DOPA-implant is obtained immersing the medical implant into (DOPA)ₓ-PEG₅-DBCO solution with the concentration of 0.005 mg/mL-1.000 mg/mL by solution immersion method for 24 h, and DOPA groups is adhered to the surface of implant through fully adhering. The non-grafted free peptides were washed off with ultrapure water to obtain polypeptideized implants, labeled as DOPA-DBCO-Ti.

### (3) Azidization of Extracellular Vesicles

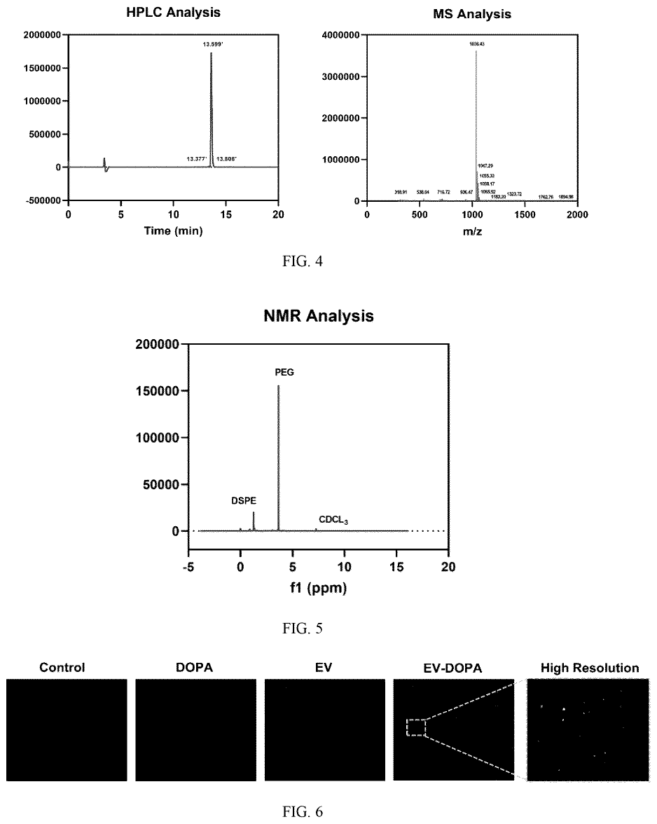

Extracellular vesicles modified by azido is prepared by mixing extracellular vesicles (EV) extracted from MSC with azide coupling reagent 1,2-distearacyl-SN-glycerol-3-hydroethanolamine phosphate (DSPE-PEG₂ₖ-Azido) in 1:100-1:10000, and it is obtained by the combined of EV and DSPE, labels as EV-DSPE-PEG₂ₖ-Azido. FIG. 5 is the NMR analysis of DSPE-PEG₂ₖ-Azido. The results reveal major groups contained in DSPE-PEG₂ₖ-Azido.

### (4) Preparation of EV-DOPA-Coated Modified Implants

The azido extracellular vesicles solution obtained in step (3) was added 3:1 (w:w) to step (2) and fully click-connected to the DOPA-adhered Ti-based material for 12 h. The specific loading of extracellular vesicles is realized by the Cycloaddition bioorthogonal click chemistry between azide groups and DBCO groups. The extracellular vesicles-modified metal-implants with immunoregulation and osteoinductive dual-functions is prepared, labeled as EV-DOPA-Ti.

### Example 3: Preparation of EV-DOPA-coated Modified Implants for Fluorescently-Label

In order to better observe the effect of implant modification, an alternative solution for the preparation of a fluorescently labeled EV-DOPA modified coating is provided in this example. It is distinguished from Example 2 in that the azide extracellular vesicles solution DSPE-PEG₂ₖ-Azido and DSPE-PEG2x-FITC containing visible fluorophore obtained in step (3) are dissolved in sterile PBS at a ratio of 3:1 (w:w) to form a mixed solution. the Azido modified EV is mixed with the extracellular vesicles derived from MSC in the process of osteogenic differentiation in molar concentration of 1:1000, after full fusion to obtain labeled as FITC/(2-Azido)-PEG₂ₖ-DSPE-EV. The labeled extracellular vesicles-modified medical implant material with immunoregulatory and osteoinductive dual-function is obtained by the reaction of FITC/(2-Azido)-PEG₂ₖ-DSPE-EV and DOPA-DBCO-Ti, labeled as FITC-EV-DOPA-Ti.

### Example 4: Surface Properties of EV-DOPA-Coated Modified Implants

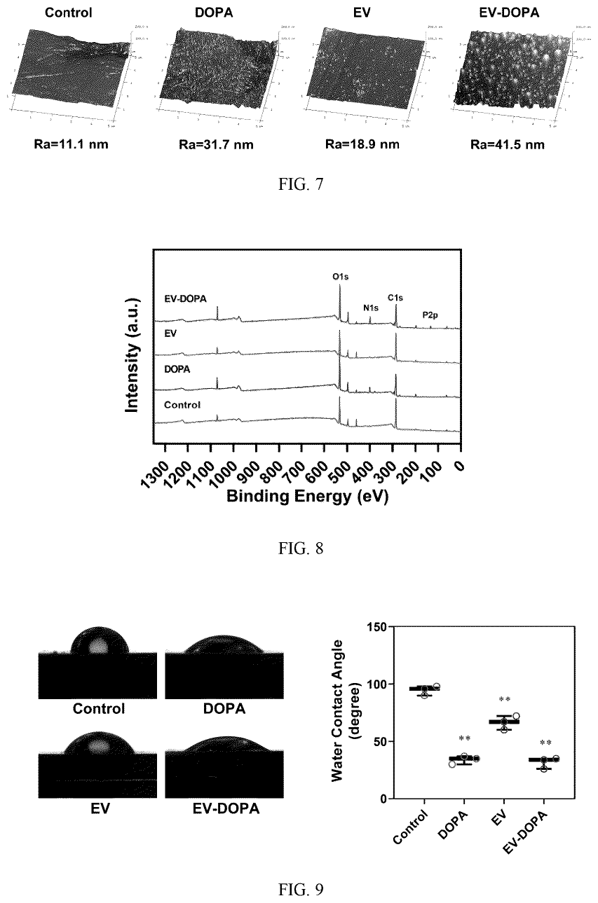

The surface properties of implants modified by EV-DOPA coating strategy and those modified by other methods are compared and verified. The EV-DOPA-coating modified implant prepared in Example 3 and other contrast implants were observed under a fluorescence microscope, respectively. The Control group is the Ti sheet soaked in ordinary PBS, the EV group is the Ti sheet modified only with extracellular vesicles, and the DOPA group is the Ti sheet modified only with DOPA group obtained in step (2) of Example 1. FIG. 6 is the images observed by fluorescence microscopy (FITC). The results show green dot fluorescence observed on the Ti surface of FITC-EV-DOPA carrying the FITC group, while no fluorescence was observed in the other groups. FIG. 7 represents the AFM images of surface topography. As can be seen from FIG. 7, the surface roughness of the DOPA group increased compared with that of the ordinary PBS-soaked Ti plates, while no significant change was observed in the EV group, while a large number of extracellular vesicles globular protrusions were observed in the EV-DOPA group. FIG. 8 is the XPS analysis of surface element composition. Comparing with the Ti plates soaked in plain PBS, the content of N element on the surface of the modified Ti-based material increased significantly. FIG. 9 shows the water contact angle images (WCA). Comparing with the Ti plates soaked in plain PBS, the water contact angle of EV-DOPA group was significantly lower, the hydrophilicity was better, and the EV was effectively modified on the Ti alloy surface without destroying the overall structure of EV.

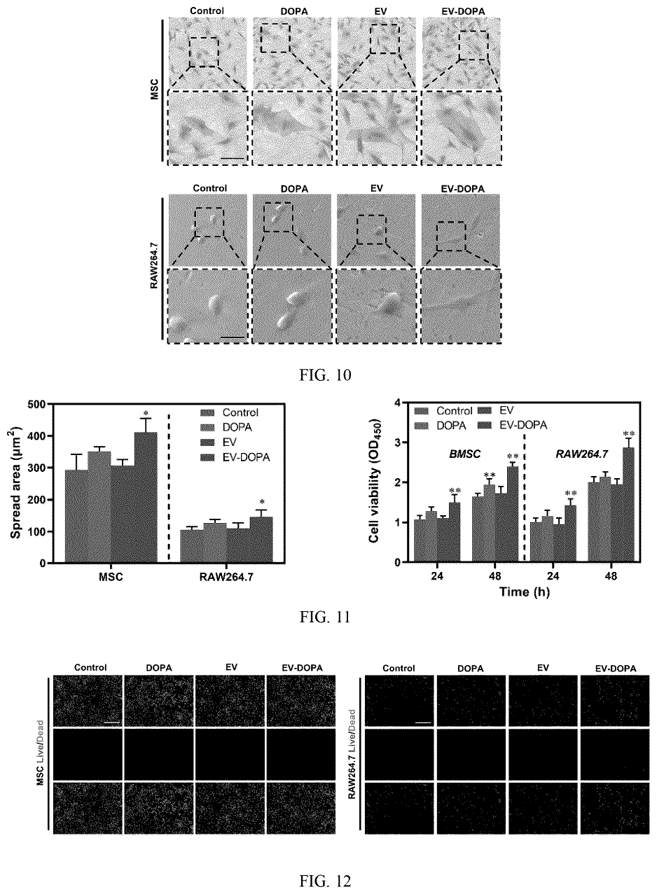

MSCs and RAW264.7 cells (homemade) were seeded on the Ti surface of each group. FIG. 10 shows the SEM of MSCs and RAW 264.7 cells. FIG. 11 shows the plots of mean cell spread analysis and cell proliferation activity assay. FIG. 12 is the Live/dead assay of MSCs and RAW 264.7 cells (**p<0.01) . As can be seen from FIG. 10-FIG. 12, comparing with the Control group, SEM images show that cells in EV-DOPA group spread more completely. No obvious dead cells are observed with live dead staining, and the EV-DOPA group had a significantly higher cell proliferation activity. The results showed that EV was successfully modified on the surface of the implant and had cell adhesion and spreading properties. These results demonstrated that the EV-DOPA-modified Ti has excellent biocompatibility.

### Example 5: Characterization of EV-DOPA Facilitated Osteogenesis In Vitro

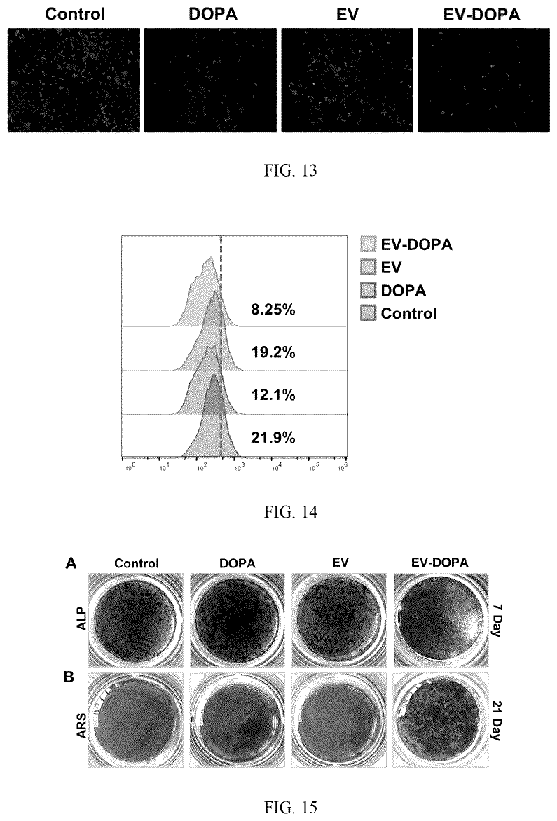

(1) Anti-oxidative stress effect of EV-DOPA SD rats aged 6-8 weeks are purchased and sacrificed under anesthesia to extract primary MSCS from long bones. The cells are inoculated on the Control group (PBS soaked Ti plates), EV group (extracellular vesicles-modified Ti plates), DOPA group (DOPA group-modified Ti plates obtained in step (2) of Example 1), and EV-DOPA group (EV-DOPA-Ti), respectively. Osteogenic induction culture is performed under high glucose and inflammatory microenvironment mimicking diabetes mellitus (DM) conditions. After 24 hours, the medium is removed by aspiration and labeled with the Biyuntian Reactive oxygen Species Detection kit according to the instructions for use. Subsequently, image data are collected using fluorescence microscopy and quantified by flow cytometry. FIG. 13 shows the fluorescence image of reactive oxygen species detection for osteogenesis of EV-DOPA. FIG. 14 shows the Quantitative flow cytometry analysis of reactive oxygen species for osteogenesis of EV-DOPA. As shown in FIG. 13 and FIG. 14, ROS fluorescence and flow detection results show that in the DM microenvironment, more ROS are produced in the Control group and the EV group. Compared with the Control group, the ROS produced in the EV-DOPA group is significantly reduced.

### (2) ALP Staining and ARS Staining

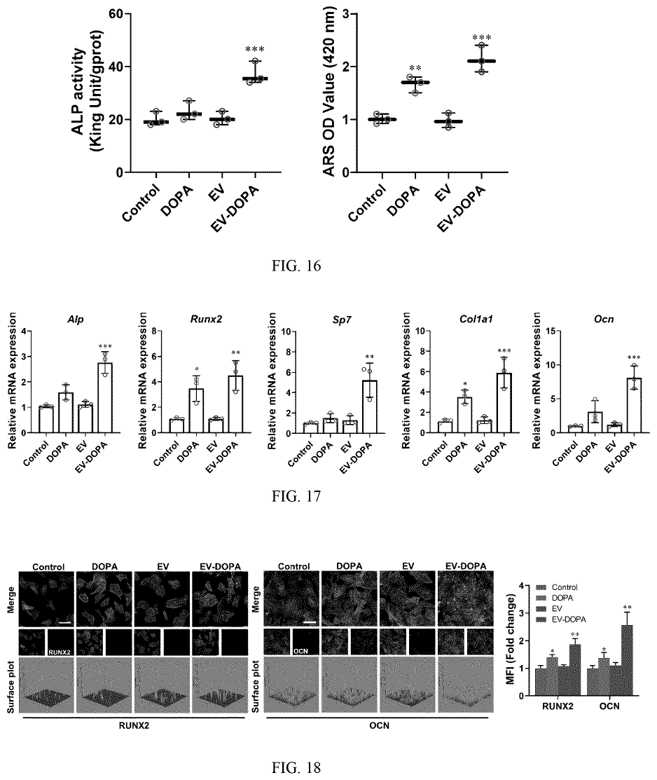

The primary MSCS are seeded in the Control group, EV group, DOPA group and EV-DOPA group, and osteogenic differentiation is induced in the DM microenvironment. At day 7 of induction, the medium is removed by aspiration, washed with PBS, stained with the alkaline phosphatase (ALP) staining kit, and images are acquired by light microscopy. The positive staining cells are blue. At day 21 of induction, the medium is removed by aspiration and, after fixation, stained with alizarin red staining (ARS) solution and images are acquired by light microscopy. FIG. 15 shows the osteogenesis staining of EV-DOPA, in which, A is for ALP staining and B is for ARS staining. FIG. 16 shows the ALP activity assay and ARS absorbance quantitative determination of osteogenesis of EV-DOPA. As shown in FIG. 15 and FIG. 16, the positive staining calcium nodules were red, and the ALP level in the Control group is low. Compared with the Control group, the oxidative stress level of MSC cells in EV-DOPA group is significantly reduced, and the ALP positive staining area and ALP activity are significantly increased (***p<0.001) . There is no significant calcium nodule formation in the Control group, while EV-DOPA group have significantly more calcium nodule positive staining and higher absorbance value (***p<0.001) compared with the Control group.

### (3) Markers of Osteogenic Differentiation

Osteogenic differentiation is characterized by RT-qPCR. Total RNA i extracted by TRIzol. The concentration and purity are evaluated by NanoDrop-2000. PrimeScript RT Master Mix is used for reverse transcription. A total of 2μl of cDNA product was used for subsequent RT-qPCR analysis using SYBRI premixture Ex TaqIM. Gene expression analysis was performed using the CFX96 Touch real-time PCR detection system.

EV-DOPA treatment promoted the expression of osteogenesis-related genes (Alp, Runx2, Ocn, Col1a1, Sp7), RUNX2, and OCN in the early and late stages of osteogenic differentiation through the detection of genes and proteins related to osteogenic differentiation. Then, RAW264.7 cells were seeded on the surface of Ti plates in different groups and cultured in a DM simulated microenvironment. FIG. 17 shows the RT-qPCR results for osteogenesis-related gene expression of EV-DOPA. FIG. 18 shows the immunofluorescence staining results of related proteins Runx2 and OCN expression. As shown in FIG. 17 and FIG. 18, RT-qPCR results show that EV-DOPA modified group have lower expression of pro-inflammatory cytokines compared with the Control group. Compared with the Control group, EV-DOPA significantly increase the gene expression levels of Alp, Runx2, Ocn, Col1a1 and Sp7 in the DM microenvironment (*p<0.05, **p<0.01, ***p<0.001). Compared with the Control group, EV-DOPA treatment significantly increase the expression of Runx2 in the early stage and OCN in the middle and late stages (*p<0.05, **p<0.01, ***p<0.001). The results demonstrate that the EV-DOPA modification inhibited macrophage differentiation to the pro-inflammatory MI type and increased the number of anti-inflammatory M2 macrophages. EV-DOPA can directly inhibit oxidation and promote bone differentiation.

### Example 6: EV-DOPA Regulates Macrophage Polarization-Mediated Immune Responses In Vitro

### (1) Changes in Macrophage Polarization Phenotype

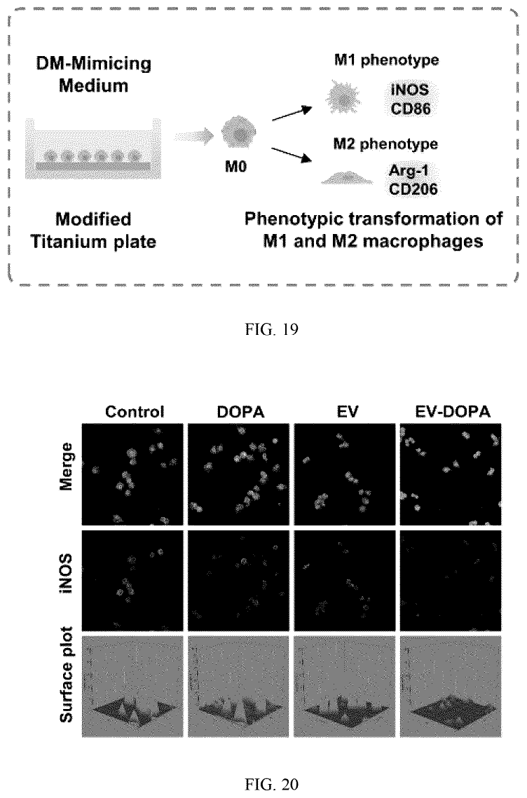

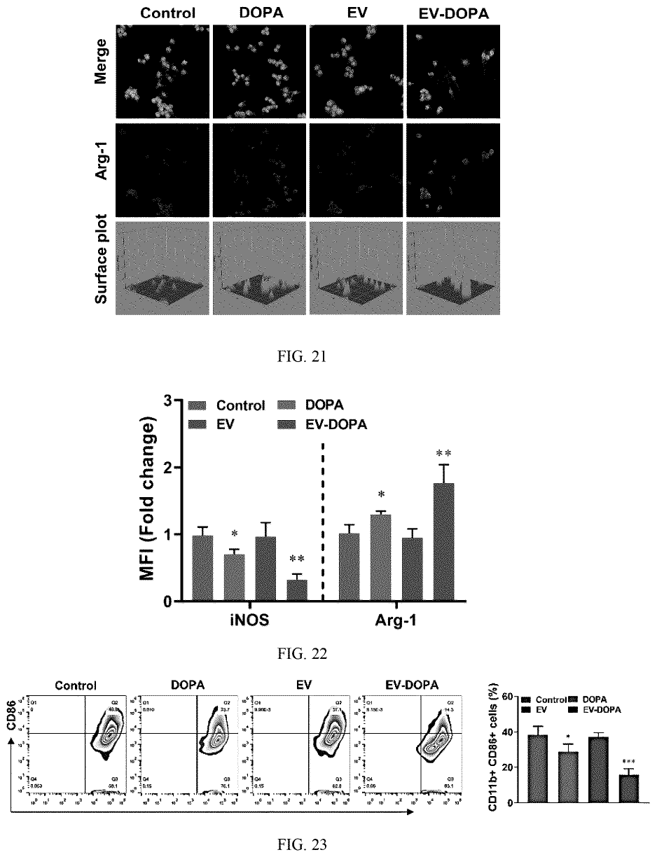

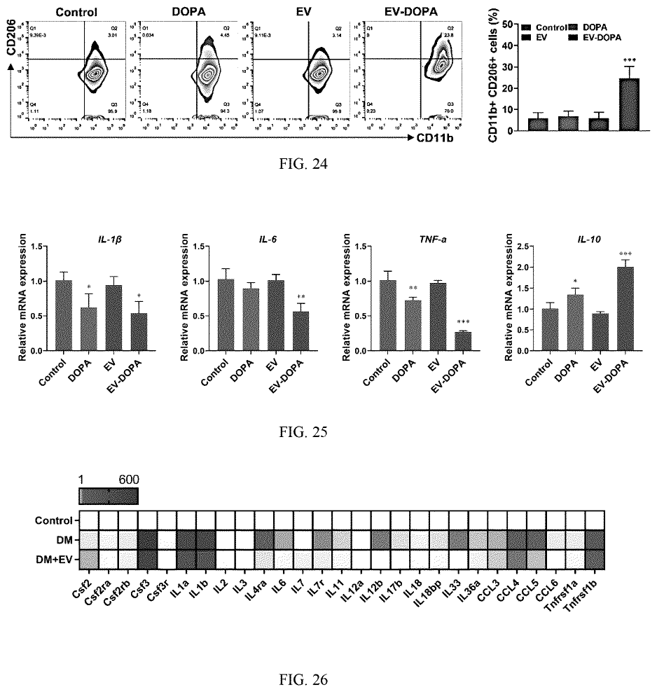

Macrophage polarization was characterized by immunofluorescence staining. FIG. 19 shows the schematic illustration of the experimental design by macrophage polarization. The experiment is as follow: the cell culture medium is removed and, after washing with PBS, 4% paraformaldehyde fixative is added and fixed on ice for 15 min. Subsequently, they are washed three times with PBS, treated with Triton solution for 10 min, and washed with PBS. Immunostaining blocking solution is added, and after blocking on ice for 1 h, the primary antibody working solution is added and incubated for 12 h at 4° C. The primary antibody is recovered, washed 3 times with PBS, and the fluorescent secondary antibody working solution is added and incubated for 1 hour at room temperature in the dark. After sealing tablets with anti-fluorescence quenching agent, images are collected under fluorescence confocal microscope. FIG. 20 shows the co-immunofluorescence staining images of iNOS and F4/80 in macrophages. FIG. 21 shows the co-immunofluorescence staining images of Arg-1 and F4/80 in macrophages. FIG. 22 shows the mean fluorescence intensity analysis images of immunofluorescent staining. As can be seen from FIG. 20-FIG. 22, the results of cellular immunofluorescence staining showed that the expression of iNOS in macrophages in DM microenvironment is higher, while the expression of Arg-1 is lower. Compared with the Control group, the expression of iNOS is decreased, while the expression of Arg-1 is increased in EV-DOPA group. FIG. 23 shows the flow quantitative analysis of M1-polarized phenotype CD86 in macrophages. FIG. 24 shows the flow quantitative analysis of M2-polarized phenotype CD206 in macrophages. Compared with the Control group, the proportion of CD11b/CD86 positive MI macrophages is significantly decreased, while the proportion of CD11b/CD206 positive M2 macrophages is increased in EV-DOPA group (*p<0.05). However, the proportion of CD11b/CD206 positive M2 macrophages increased (*p<0.05, **p<0.01, ***p<0.001), **p<0.01, ***p<0.001).

### (2) Inflammatory Cytokines

Inflammatory cytokines are characterized by RT-qPCR. The specific implementation is the same as step (3) in Example 4. FIG. 25 shows the RT-qPCR of inflammatory cytokines. The results show that EV-DOPA significantly reduced the expression of pro-inflammatory cytokines IL-1β, IL-6 and TNF-α and increased the expression of anti-inflammatory IL-10 (*p<0.05, **p<0.01, **p<0.001) in DM microenvironment.

FIG. 26 shows the expression analysis of inflammatory factors by high-throughput sequencing. It shows that EV-DOPA can significantly reduce the expression of most inflammation-related factors in DM microenvironment as analyzed by high-throughput sequencing results. These results confirm that EV-DOPA can regulate the immune responses mediated by macrophage polarization.

### Example 7: EV-DOPA Indirectly Promoted Osteoblast Differentiation through Immune Regulation In Vitro

### (1) ALP Staining and ARS Staining

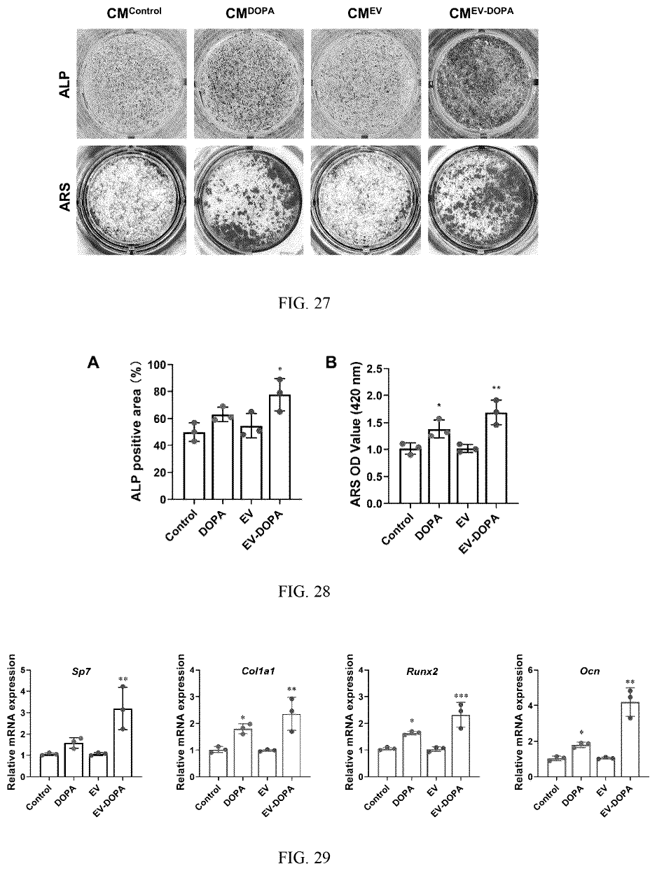

ALP and ARS staining procedures are the same as in step (2) of Example 4. The EV-DOPA-modified prosthesis is then cocultured with conditioned medium prepared from macrophage culture. FIG. 27 shows that staining under EV-DOPA-regulated immune conditions, in which, A is for ALP staining and B is for ABS staining. FIG. 28 shows the ALP activity assay and ARS absorbance quantitative determination of EV-DOPA-regulated immune conditions. FIG. 27 and FIG. 28 showed that the ALP level in CMControl group was low. Compared with the CMControl group, the ALP positive staining area and ALP activity in the CMEV-DOPA group are significantly increased (*p<0.05). No calcium nodules are observed in the CMControl group, while the CMEV-DOPA group had significantly more calcium nodules and higher optical density (** p<0.01) .

### (2) Markers of Osteogenic Differentiation

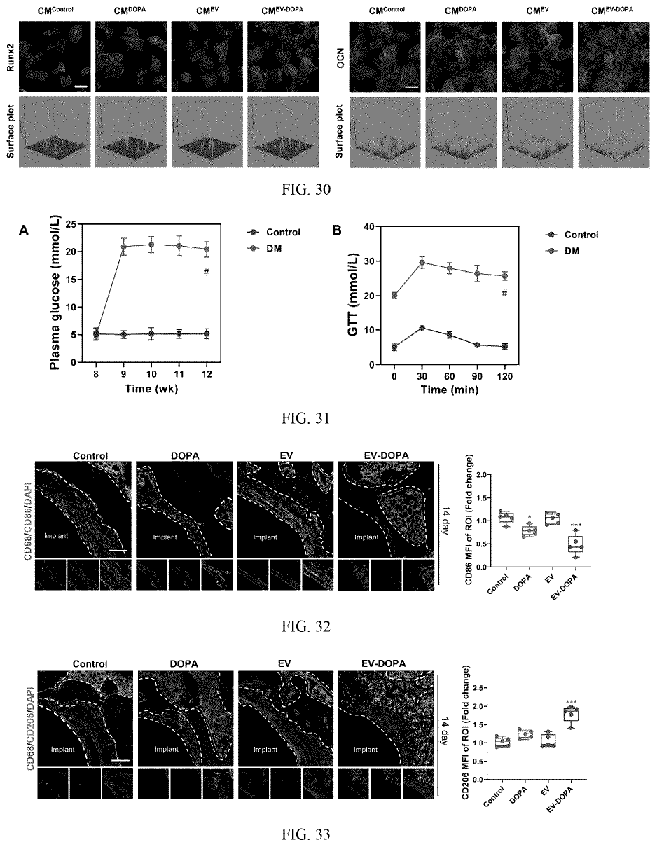

RT-qPCR test is used to characterize the osteogenic differentiation, and the specific implementation is the same as step (3) in Example 4. FIG. 29 shows the RT-qPCR results for EV-DOPA-regulated immune conditions. Compared with the CMControl group, the gene expression levels of Alp, Runx2, Ocn and Col1a1 are significantly increased in DM microenvironment. FIG. 30 shows the immunofluorescence staining results of related proteins Runx2 and OCN expression under EV-DOPA-regulated immune conditions. As show in FIG. 30, compared with the CMControl group, the expression of Runx2 in the early stage and OCN in the middle and late stage increased in the CMEV-DOPA group (*p<0.05, ** p<0.01). These results suggest that EV-DOPA is able to promote osteogenic differentiation by regulating the immune response mediated by macrophage polarization.

### Example 8: EV-DOPA Promotes Periprosthetic Osseointegration in DM Rats In Vivo

(1) Animal model

Streptozotocin (STZ; Sigma Aldrich) induced diabetes model is used. Five-week-old male SD rats weighing 100 g are adaptively fed for 1 week. STZ solution is prepared by adding 1% (w/v) of STZ into citric acid buffer (pH 4.2-4.5). After fasting for 12 hours, the rats are injected with STZ at a dose of 35 mg/kg by intraperitoneal injection. Tail vein blood samples are obtained weekly for non-fasting blood glucose (BGL) analysis. Successful modeling is defined as BGL>16.7 mM. Glucose tolerance test (GTT) is performed to test islet function. Rats are fasted for 12 h before testing. Blood is collected from the tail vein, and blood glucose is measured before intraperitoneal injection of glucose solution (1 g/kg) as a control value. Blood glucose is measured at 30, 60, 90 and 120 min after injection to observe glucose tolerance level. The results show that the diabetic model had been successfully established.

Ti rod implantation is performed 3 weeks after STZ injection. The incision is made in the midline of the rat knee joint to expose the distal femur. A hole is drilled in the center, and the Ti rods (diameter: 1.5 mm, length: 10 mm) of each group are implanted in the distal femur parallel to the longitudinal axis of the femur. According to the different modification methods of the implant materials, the animals are divided into four groups: PBS immersion group (Control), DOPA modification group (DOPA), EV simple immersion group (EV) and EV-DOPA modification group (EV-DOPA).

During the modeling period, the animals in each group could move freely in the cage, eat normally, increase water intake and urine output, and there is no significant increase in body weight. There is no significant change in mental status. FIG. 31 shows the blood glucose monitoring chart of DM rat model, in which, A is the continuous blood glucose monitoring of various week-aging rats and B is the glucose tolerance test. One week after modeling, the rats show a significant increase in blood glucose. There is no infection or inflammatory reaction such as redness, swelling and exudation of the incision after implantation, and all patients healed in one stage. No animal died during the experiment.

### (2) Early Changes in Macrophage Polarization in Prosthesis Periphery

Immunohistochemical fluorescence staining is used to observe the polarization of macrophages around the prosthesis, as follows:
1) Dewaxing and hydration

Before deparaffinization, the sections are baked in a thermostat at 60° C. for 30 min. The slices are immersed in xylene for 10 min, and then immersed for another 10 min after replacing xylene. The cells are immersed in absolute ethanol for 5 min. They are then soaked in 95% ethanol for 5 min. The cells are soaked in 70% ethanol for 5 min.
2) Antigen retrieval

Enzyme digestion method: 0.1% trypsin is commonly used. The slices are also preheated to 37° C. before trypsin use. 0.2 ml of digestion solution is dropped on each slice to cover the complete tissue, and the slices are digested in a 37° C. incubator for about 5-30 min. During the proceed, it should keep away from light.
3) Immunohistochemical staining

The cells are washed 2 to 3 times with PBS for 5 min each. Normal goat serum blocking solution is added drop by drop at room temperature for 20 min. Toss off excess liquid. 100 μl of antibody I is dropped and incubated at 4° C. overnight or 37° C. for 1 hour. The cells are washed 3 times with PBS for 5 min each. 40-50 μl of antibody II is dropped and left at room temperature or 37° C. for 1 hour. The cells are washed 3 times with PBS for 5 min each. 20 μl DAPI-containing anti-fluorescence quenching sealant is dropped, and the sealant is covered with a cover glass and observed under a fluorescence microscope. FIG. 32 shows the co-immunofluorescence staining images and mean fluorescence intensity analysis of CD86. FIG. 33 shows the co-immunofluorescence staining and mean fluorescence intensity analysis images of CD206. FIG. 32-33 shows that two weeks after Ti rod prosthesis implantation, the expression level of CD86, a marker protein of M1 macrophages, is higher in the periprosthetic tissues of PBS Control group, and the number of CD68/CD86 double positive cells was more, while the number of CD86/CD206 positive cells (M2 macrophages) was less, and the expression of M2 macrophages was low. In contrast, EV-DOPA treatment significantly reduced the expression of CD86 and the number of CD68/CD86 positive M1 macrophages, while increased the number of CD86/CD206 positive M2 macrophages (*p<0.05, **p<0.01, ***p<0.001). These results indicate that the surface functionalization of EV-DOPA prosthesis can regulate the polarization phenotype of periprosthetic macrophages.

### (3) Micro-CT Detection

The hind limbs of mice are scanned and analyzed using a high-resolution micro-CT SkyScan 1076 produced by SkyScan, Belgium. Specimens are removed from the fixative and allowed to dry before scanning. Each specimen is placed in Micro-CT test tube cup, 5 specimens at a time, and each specimen is separated by foam plastic sheet. The specimens should be arranged neatly to avoid touching the test tube wall. The scanning parameters are set as follows: voltage 50 kV, current 800 μA, scanning time 1750 ms, spatial resolution 18 μm. After completion of scanning, SkyScan 1176 software is used for three-dimensional reconstruction of the mouse ankle joint. CT Analyzer software is used to analyze the following parameters: bone mineral density (BMD; mg/cm3). Bone volume fraction (BV/TV; %); Trabecular separation (Tb.Sp; μm) and trabecular bone number (Tb.N).

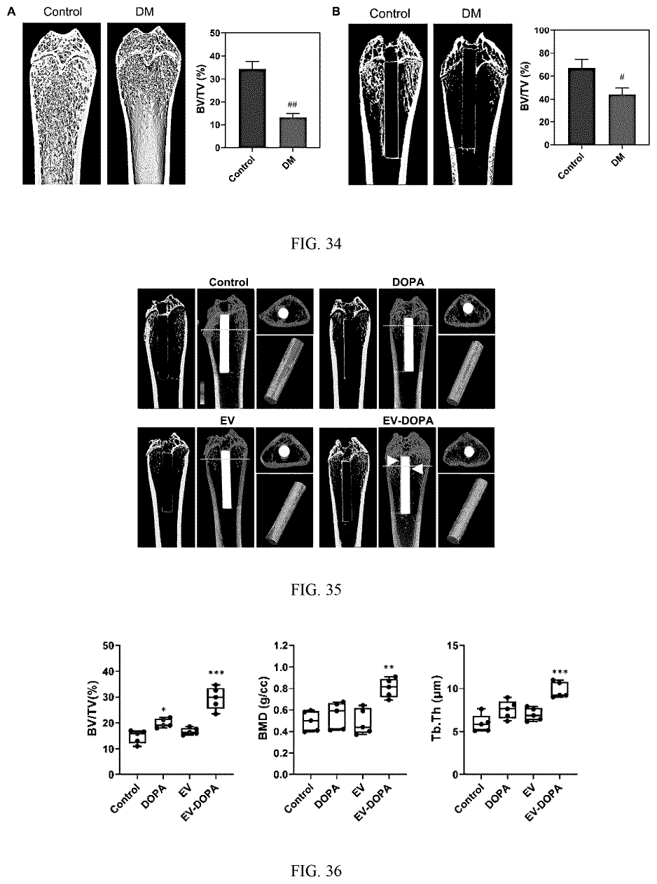

Micro-CT can be used to scan the bone tissue of experimental rats, and three-dimensional image reconstruction and quantitative analysis can accurately describe the bone mass and bone microstructure, so as to determine the changes of bone parameters and the osseointegration around the prosthesis in the rat model. FIG. 34 shows the three-dimensional reconstruction images of bone mass and bone volume fraction analysis of normal rats and DM rats before and after Ti rod implantation, in which, A is before Page Ti rod implantation and B is after Ti rod implantation. Compared with healthy rats, the BMD of long bones in DM rats decreased. The osseointegration ability of the Ti rod prosthesis was insufficient (#p<0.05, ##p<0.01). Bone three-dimensional reconstruction and bone parameter analysis were performed on different groups of experimental tissues. FIG. 35 shows the Micro-CT bone three-dimensional reconstruction image of EV-DOPA on periprosthetic osseointegration in DM rat. FIG. 36 shows the bone volume fraction analysis, bone mineral density analysis and trabecular thickness analysis of EV-DOPA on periprosthetic osseointegration in DM rat. FIG. 35 and FIG. 36 show that the bone mass around the prosthesis in the Control group is lower, and the bone mineral density (BMD), bone volume fraction (BV/TV), and trabecular bone number (Tb.N) are all at a low level. In EV-DOPA group, periprosthetic bone mineral density, bone volume fraction and trabecular number were significantly higher than those in Control group (*p<0.05, **p<0.01, ***p<0.001). These results demonstrate the EV-DOPA could effectively improve the periprosthetic bone mass.

### (4) Histological Staining

Hematoxylin and eosin (H&E) staining and toluidine blue staining are used to observe the histomorphology. H&E staining is performed as follows:
1) The femoral tissue samples are decalcified by EDTA decalcification solution, trimmed, and embedded in paraffin. The paraffin-embedded specimens are sliced using a tissue microtome, and finally made into paraffin sections with a layer thickness of 6 um.
2) Paraffin sections are deparaffinized with xylene (10 minx3 times) and then immersed in 100%, 95%, 90% and 85% ethanol solution in water, with 5 min for each passage. After rinsing with distilled water for 3 min, the cells are stained with hematoxylin solution for 5 min and rinsed with tap water for 5 min. The cells are differentiated in 1% hydrochloric acid alcohol solution for 60 s, and then rinsed with tap water for 1 min. Blue is returned to 10% ammonia solution for 60 seconds, and then rinsed with tap water for 1 min. The cells are counterstained with 1% eosin solution for 3 min and rinsed with tap water for 1 min. Conventional gradient ethanol solution and xylene are dehydrated, transparent, and sealed with resin.

Toluidine blue staining is performed as follows: after hard tissue sectioning, toluidine blue staining solution is diluted with water (the dilution was determined according to the desired staining degree, starting from 1:100 dilution according to experimental experience). The slides are soaked briefly in the diluted staining solution, and then soaked several times in water, followed by table-pressing fixation according to the selected method.

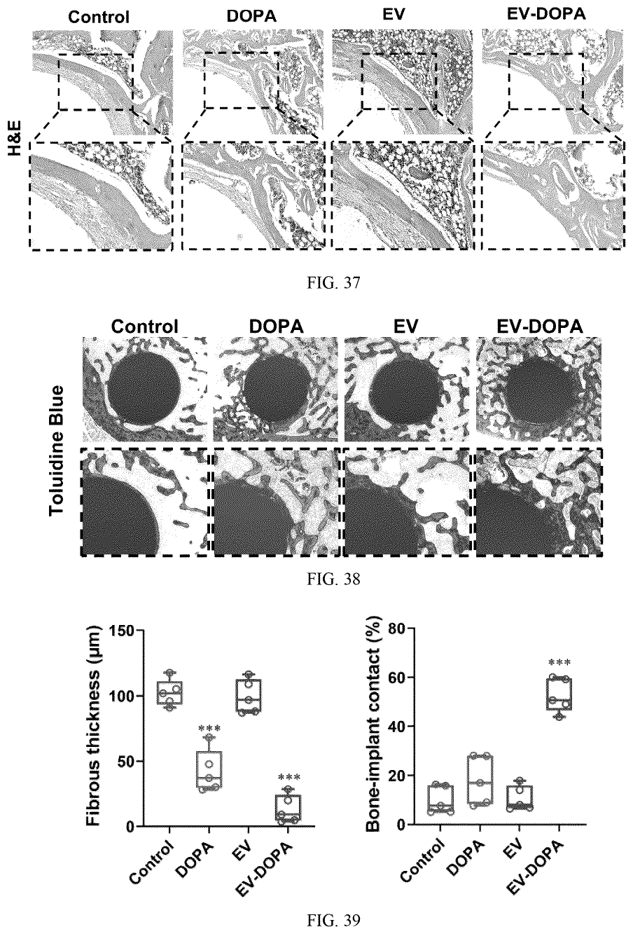

FIG. 37 shows the H&E staining of EV-DOPA on periprosthetic osseointegration in DM rat. FIG. 38 shows the toluidine blue staining images. FIG. 39 shows the analysis of fibrous layer thickness and bone-prosthesis contact ratio. As observed from FIG. 37-FIG. 39, at 8 weeks after prosthesis implantation, H&E staining results show that obvious annulus fibrosus appeared in the Control group and the EV group. Periprosthetic annulus thickness is significantly reduced in EV-DOPA group compared with Control group (***p<0.001) . Toluidine blue staining showed that the amount of new bone around the prosthesis is less in the Control group and EV group. Compared with the Control group and EV group, the amount of new bone around the prosthesis is significantly increased in EV-DOPA group (***p<0.001) . These results that EV-DOPA inhibits periprosthetic annulus fibrosus formation and promotes new bone formation and osseointegration on the prosthetic surface.

### (5) Osteogenic Markers Detection

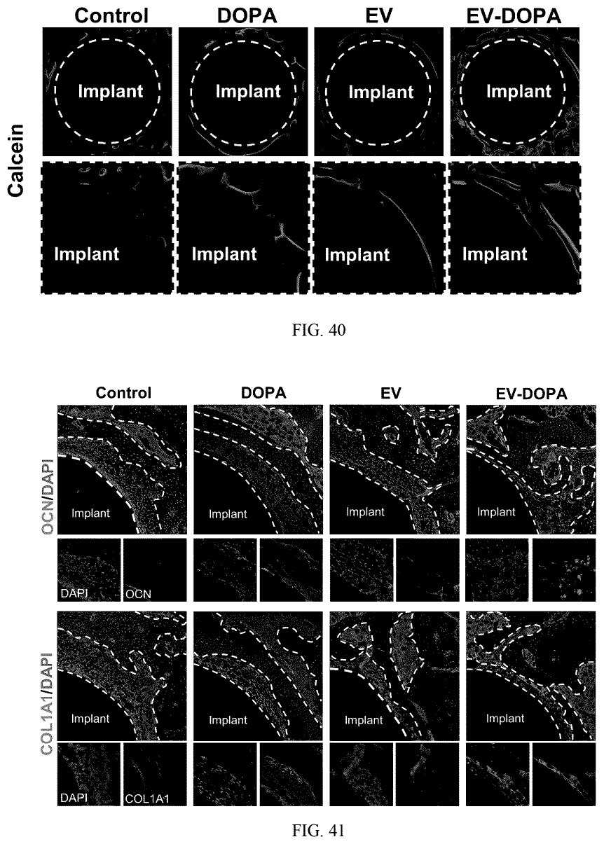

Calcein fluorescence staining is used characterize the osteogenic properties of materials. FIG. 40 shows the calcein fluorescence images of EV-DOPA on osteogenic markers of the prosthesis periphery in DM rats. The results of calcein fluorescence showed that in the Control group and the EV group, the calcein fluorescence area around the prosthesis was small, and the gap was not obvious, suggesting that the bone formation was low. Compared with the Control group, more calcein fluorescence appeared around the prosthesis in the EV-DOPA group, and the fluorescence gap was significantly widened. FIG. 41 shows protein immunofluorescence staining images of OCN and COL1A1. As can be seen in FIG. 41, immunohistochemical staining of the osteogenic differentiation markers OCN and COL1A1 showed that the expression of the osteogenic differentiation markers is lower in the Control group, while EV-DOPA significantly increase the expression of OCN and COL1A1 in the EV-DOPA group. These results demonstrate that the functional modification of EV-DOPA can effectively promote osteogenic differentiation around the prosthesis in the DM microenvironment.

All data are analyzed by SPSS11.0 statistical software and presented as mean±standard deviation (SD) (x̅±s). One-way analysis of variance (ANOVA) was used to compare data between more than two groups. LSD and Dunnett-t tests were used for pairwise comparison under the condition of homogeneity of the population variance. p<0.05 is considered statistically significant.

The above examples are preferred implementations of the present disclosure, but the present disclosure is not limited to the above implementations. Any obvious improvement, substitution, or modification made by those skilled in the art without departing from the essence of the present disclosure should fall within the protection scope of the present disclosure.

What is claimed is:

1. An extracellular vesicles-modified metal-implant with immunoregulatory and osteoinductive dual-functions, wherein
the immunoregulatory and osteoinductive dual-functions are realized by the extracellular vesicles-modified medical implant through a method of mussel-derived adhesion peptides and azidylated extracellular vesicles on a surface of a Ti-based material;
the extracellular vesicles-modified medical implant is coated with mussel-derived (DOPA)ₓ-PEG₅-DBCO peptides that can undergo a click reaction with azide groups linked with extracellular vesicles on the surface of the Ti-based material: and
a stable and nontoxic modification of the extracellular vesicles is achieved through a cycloaddition bioorthogonal click chemical reaction between azidinated extracellular vesicles DSPE-PEG₂ₖ-Azido and (DOPA)ₓ-PEG₅-DBCO.

2. (canceled)

3. A preparation method of an extracellular vesicles-modified metal-implant with immunoregulatory and osteoinductive dual-functions comprising the following steps:
(1) synthesizing a mussel-derived peptide from a copper-free click chemical dibenzo-cycloalkyene DBCO, acetone and Fmoc protected L-DOPA amino acids Fmoc-DOPA(acetonide)-OH by a solid-phase peptide synthesis method marked as (DOPA)ₓ-PEG₅-DBCO, wherein a structure of the amino acid sequence of the L-dopa amino acid is simplified as: Ac-[(DOPA)-G]_{x/2}-K[(PEGs)-(Mpa)-(Mal-DBCO)]-[(DOPA)-G]_{x/2}, in which, x is a number of repeat units of DOPA amino acids, x is selected from 2, 4, 6, and 8; an overall chain length of mussel-derived peptides is 5, 9, 13, or 17 amino acid groups, and a linkage mode is linear;
(2) fully immersing the medical implant into a (DOPA)ₓ-PEG₅-DBCO solution obtained in the step (1), wherein a DOPA-implant with DOPA groups sufficiently adhering to the implant surface is achieved;
(3) mixing of an EV derived from MSC with Azido coupling reagent 1,2-distearyl-Sn-glycerol-3-hydroethanolamine phosphate DSPE-PEG₂ₖ-Azido, wherein the azidinated extracellular vesicles obtained by conjugating the EV with DSPE are marked as EV-DSPE-PEG₂ₖ-Azido;
(4) obtaining extracellular vesicles-modified metal-implants with dual-functions of immunomodulatory function and osteoinductive activity by immersing the DOPA-implant obtained in the step (2) into the EV-DSPE-PEG₂ₖ-Azido solution obtained in the step (3), wherein a specific loading of the extracellular vesicles is realized by the cycloaddition bioorthogonal click chemical reaction between azide groups and DBCO groups.

4. (canceled)

5. The preparation method according to claim 3, wherein in step (2) the medical implant is a Ti alloy material, and is preferably a Ti plate, a Ti rod, or a Ti nail.

6. The preparation method according to claim 3, wherein in step (2) a concentration of the (DOPA)ₓ-PEG₅-DBCO solution-in is 0.005 mg/mL-1.000 mg/mL, and the full immersion is conducted for 12-36 h.

7. The preparation method according to claim 3, wherein in step (3) a ratio by mass of the EV to DSPE-PEG₂ₖ-Azido is 1:100-1:10000.

8. The preparation method according to claim 3, wherein in step (4) the concentration of EV-DSPE-PEG₂ₖ-Azido solution is 0.01-1.0 mg/ml, and the immersion is conducted for 6-24 h.

9. A artificial prosthesis implant material comprising the extracellular vesicles-modified metal-implant with the immunoregulatory and osteoinductive dual-functions according to claim 1.

10. The artificial prosthesis material according to claim 9, wherein the artificial prosthesis material is used for a diabetic patient.

11. An artificial prosthesis implant material prepared by the method of an extracellular vesicles-modified metal-implant with immunoregulatory and osteoinductive dual-functions according to claim 3.

12. The artificial prosthesis material according to claim 11, wherein the artificial prosthesis material is used for a diabetic patient.
